(19) United States
(12) Patent Application Publication
(10) Pub. No.: US 2002/0151747 A1
(43) Pub. Date: Oct. 17, 2002

(54) Method for the manufacture of acrylic or methacrylic acid

(76) Inventors: Jerry D. Unruh, Manitou Springs, CO (US); Norma Jean Diaz, Bishop, TX (US); Robert Ray Molina, Corpus Christi, TX (US); Phillip Sidney Snyder, Corpus Christi, TX (US); Kenneth Allen Windhorst, Portland, TX (US)

Correspondence Address: James J. Mullen, c/o Celanese Ltd., P.O. Box 9077, Corpus Christi, TX, 78469-9077, US

(21) Appl. No.: 09/833,945

(22) Filed: Apr. 12, 2001

(51) Int. Cl. C07C 51/21

(52) U.S. Cl. 562/546

(57) ABSTRACT

A method for the manufacture of acrylic acid or methacrylic acid by the oxidation of propylene, acrolein, or isobutylene by:

a) reducing palladium acetate to unsupported palladium with propylene in an oxygen-free single or two phase aqueous solution containing as a co-solvent a maximum concentration of a C₂-C₆ carboxylic acid or C₃-C₆ ketone in a reactor adapted for continuous phase production,

b) thereafter adding air and propylene, acrolein, or isobutylene in a continuous manner,

c) recovering the acrylic acid or methacrylic acid formed, and

d) recycling the solvent to the reactor.

### BACKGROUND OF THE INVENTION

Acrylic acid is manufactured commercially by the vapor-phase oxidation of propylene or acrolein with an oxygen-containing gas. The oxidation of propylene is carried out at a temperature of from about 425°-450° C. in the presence of water vapor or steam and a catalyst comprising predominately Mo—Bi—W oxide. A second stage oxidation is then carried out at lower temperature with a Mo—V catalyst to convert the mainly acrolein to acrylic acid. These oxidations are carried out generally at atmospheric pressure. The reaction is quenched in water and the acrylic acid is recovered therefrom by distillation.

Several “new generation” methods for the oxidation have been proposed. The most promising is to carry out the oxidation in liquid phase utilizing a palladium catalyst.

The methods for the manufacture of acrylic acid from the palladium-catalyzed oxidation of propylene using an oxygen-containing gas in water or an aqueous medium have been previously disclosed in the literature. Disclosures include methods to activate the catalyst and co solvents to improve the solubility of the components in the aqueous solution.

Literature which specifically discloses methods to activate the catalyst and use of the catalyst to oxidize propylene to acrylic acid in aqueous solution include:

David and Estienne, U.S. Pat. No. 3,624,147, which discloses the oxidation in water using various forms of palladium metal including unsupported palladium. The supported palladium is disclosed as being supported on silica gel, silica-alumina and carbon. The oxidation was carried out at 50-60 bar pressure.

Various forms of palladium catalyst useful for the reaction are discussed by Seiyama et. al., Catalytic Oxidation of Olefins over Metallic Palladium Suspended in Water, J. Catalyst, 173 (1972). Unsupported Pd catalyst is manufactured by the reduction and activation of palladium chloride.

Exposure of palladium on carbon to propylene in a oxygen free atmosphere prior to its use as an oxidation catalyst for the oxidation of propylene to acrylic acid in water containing the free radical inhibitor BHT was disclosed by Lyons, Dependence of Reaction Pathways and Product Distribution on the Oxidation State of Palladium Catalysts for the Reactions of Olefinic and Aromatic Substrates with Molecular Oxygen in Oxygen Complexes and Oxygen Activation by Transition Metals, Martell and Sawyer, ed., Plenum Press, (1988).

Lyons and Suld, EP 0 145 467 B1, discloses activating palladium metal on a carbon or alumina support to an oxygen-free atmosphere of propylene at from 60°-150° C. for from 10-120 minutes at 1-100 atmospheres prior to using the catalyst for the oxidation of propylene to acrylic acid in an aqueous solution. This catalyst at 1-10 atmospheres and 25°-85° C. oxidized propylene in an aqueous solution.

Suld and Lyons, EP 145 468 A2, discloses the use of the above catalyst to manufacture acrylic acid in an aqueous solution containing a surfactant and a co-surfactant. The surfactant was sodium dodecyl sulfate and the co-surfactant was a C₃-C₄ alkyl alcohol.

Lyons, EP 145 469 B1, discloses the use of the catalyst of Lyons and Suld above in the oxidation of propylene to acrylic acid in an aqueous solution containing a free radical inhibitor, i.e., BHT.

Pasichnyk et. al., Oxidation of Propylene to Acrylic Acid and its Esters Catalyzed by Palladium Giant, Mendeleev Commun. (1994), (1), 1-2 [CAPLUS Document No. 120: 245831] discloses the oxidation of propylene using giant crystals of palladium.

Hinnenkamp, U.S. Pat. No. 4,435,598, discloses the use of Pd/C catalyst in aqueous solution using hydroquinone.

### SUMMARY OF THE DISCLOSURE

The method of this invention manufactures acrylic acid and methacrylic acid from the palladium-catalyzed oxidation of propylene or isobutylene respectively in an aqueous solution utilizing an oxygen-containing gas. This invention also encompasses the oxidation of acrolien to acrylic acid.

The method of this invention differs from the prior art methods disclosed above in that (1) the palladium catalyst is finely divided unsupported metal manufactured in situ by the reduction and activation of palladium acetate in one step. The reduction is carried out with propylene in an oxygen-free aqueous solution containing a C₂-C₆ carboxylic acid or C₃-C₆ ketone. Propylene or isobutylene and an oxygen-containing gas are then introduced into the mixture in a continuous manner and the resulting aqueous acid is continuously removed. The acid is separated by distillation in a manner well known to the art and discussed in the prior art references. The aqueous residue is then continuously returned to the reactor to maintain a constant level in the reactor.

### DETAILED DESCRIPTION OF THE INVENTION

According to the method of the present invention, acrylic acid and methacrylic acid can be manufactured in high conversion and yield by carrying out the oxidation of propylene and isobutylene respectfully in an aqueous solution in the presence of palladium catalyst. The David, Estienne patent, U.S. Pat. No. 3,624,137, discloses the use of an unsupported palladium metal catalyst. The catalyst was, however, a commercial palladium metal catalyst. The result of the use of unsupported catalyst in comparison with the supported catalysts as disclosed in the patent provides a product high in saturated acids. Other prior art discloses the use of palladium supported on carbon, alumina, and other supports as the catalyst. The prior art discloses the use of water and of aqueous solutions containing free radical inhibitors, as for example BHT as the medium for the oxidation. The prior art also discloses the presence of lower alkyl alcohols as additives to the aqueous solution during the oxidation reaction to increase the solubility of the reactants.

This invention pertains to a method for the manufacture of acrylic acid and methacrylic acid which comprises: a) continuously reacting oxygen with the precursor gaseous hydrocarbon in the presence of an unsupported palladium catalyst suspended in an aqueous solvent system containing as a co-solvent a C₂-C₆ carboxylic acid or C₃-C₆ ketone, b) recovering the acrylic acid formed, and d) recycling the aqueous solvent to the reactor. The catalyst reduction is efficiently carried out with propylene. The solvent system may or may not be a single-phase system. Ideally, the solvent system is a single-phase system containing a saturation concentration of co-solvent.

According to one embodiment of this invention, the palladium catalyst is prepared in the oxidation reactor prior to the oxidation reaction. The preparation of the catalyst involves dissolving palladium acetate in the single or two-phase solvent, discussed below, flushing the solution and vessel with a gas inert to the reaction, and contacting the solution with propylene in a vigorous manner, as for example by stirring, rapid agitation, or by a similar method. The inert gas may be nitrogen, helium, argon, krypton, or the like inert gases. Typically the reaction is complete in about 1-2 hours at 60°-90° C. at a pressure of about 1-50 bar. One skilled in the art, however, would recognize that the temperature could be increased or decreased as needed. Temperature ranges of from 50°-150° C. can be used. Reaction times of 0.5-5 hours might be needed to complete the reaction at other temperatures. According to the Law of Mass Action, the higher temperatures will allow the reaction to be completed in a shortened amount of time, but will lead to a greater amount of undesired products. It has been found that there is no advantage to carry out the reaction at elevated pressures. Ambient atmospheric pressure is sufficient.

If the reduction is carried out in a manufacturing process separate and apart from the acid production process, care must be taken to separate and store the freshly reduced catalyst, particularly to separate and store the catalyst away from oxygen or an oxidizing atmosphere. Use of freshly reduced catalyst is to be desired since catalyst stored for extended periods tends to lose activity to the manufacture of the desired product. Catalyst prepared in the acid manufacturing equipment and used without further manipulation is to be preferred, although the use of stored catalyst is not outside the invention and without the claimed method of this invention. The catalyst is generally stored under water and is separated and dispersed by immersion in an ultrasonic bath prior to use. The catalyst tends to clump and must be stirred or agitated rapidly in order to avoid clumping which reduces the activity.

The acid manufacturing reaction of this invention is carried out continuously by passing propylene and an oxygen-containing gas into a reactor containing the catalyst in an aqueous solvent containing an appropriate amount of a co-solvent as defined hereinbelow and removing the product acid by continuously separating the liquid component from the solid catalyst, removing a portion of the catalyst-free solvent, separating the product acid therefrom, and recycling the solvent. Temperatures in the reactor are preferably from about 50° C. to about 150° C. and pressures are from about atmospheric to about 50 bar. The molar ratio of propylene to oxygen is preferably above 1:1, but is most preferably from about 1:1 to about 1:5. Oxygen-containing gases may be pure oxygen or mixtures of oxygen with other gases that are inert to the reaction. Examples of such gases are air, and oxygen-containing mixtures, as for example oxygen-nitrogen, oxygen-helium, oxygen-argon, and the like mixtures. A co-solvent of a C₂-C₆ carboxylic acid or C₃-C₆ ketones is advantageous to the solubility of the components in the catalyst preparation and in the oxidation reaction. The preferred acids include acetic acid, propionic acid, butryic acid, valeric acid, and hexanoic acid. Acids with a lower boiling point are effective in promoting the reaction but are more difficult to separate from the acrylic acid in the reaction mixture, needlessly complicating the separation of the acrylic acid during separation and purification. Higher fatty acids are detrimental to the reaction and should be avoided. Ketone co-solvents are preferred in the manufacture of methacrylic acid. The preferred ketones include acetone, methyl ethyl ketone, methyl isobutyl ketone, and the like. The most preferred co-solvent for the manufacture of methacrylic acid is methyl isobutyl ketone. The most preferred solvent for the manufacture of acrylic acid is valeric acid. Alcohols are to be avoided as co-solvents to prevent a possible reaction with the formed acrylic acid or methacrylic acid to manufacture acrylic and derivative or acetic and derivative ester by-products and oxidation to acids and esters, which would reduce the yield of desired product and would complicate the separation process.

Acrolein, being an intermediate in the process of manufacture of acrylic acid from propylene, acrolein can also be oxidized by the aforedescribed process.

Separation of the product from the catalyst is accomplished by methods well known in the art, as for example, by filtration, decantation, centrifugation, distillation, and the like. In the continuous mode of operation of the oxidation reaction of this invention, separation is ideally accomplished by use of a filter. The filter may be internal to the reaction vessel, as for example a candle filter, or external to the reactor. Separation of the product acrylic acid from the solvent is carried out by distillation or decantation and distillation. The solvent is then recycled to the reactor.

Preferred separation of acrylic acid from valeric acid is by fractional distillation. The water forms an azeotrope with several of the low-boiling byproducts and is removed first. The acrylic acid then is recovered, and the higher-boiling valeric acid is returned to the reactor. No additional water is added with the recycle since the byproduct water in the reactor is sufficient to act as the aqueous solvent.

Preferred separation of methacrylic acid from methyl isobutyl ketone is accomplished by decantation of the organic layer, fractional distillation of the recovered organic layer, and return of the ketone to the reactor. Again no additional water need be added with the recycled solvent.

Several batch experiments were run utilizing propylene in order to determine the efficacy of the co-solvents and the appropriate time and temperature regimes. In the propylene batch procedure, a mixture of 30 g liquid, and unsupported palladium catalyst prepared from 0.75 g Pd(OAc)₂ as described hereinbelow and kept moist after preparation was placed in a 100 ml. Parr autoclave equipped with a stirrer. The autoclave was flushed twice with propylene during which the stirrer was activated. The autoclave was then pressured with propylene to a pressure of 2.75 bar and stirring was begun at 1200 rpm. External heating was used to cause the contents to attain a temperature of 80° C. at which point 29 bar air was added. A pressure drop was noted immediately and the reaction was terminated when the pressure drop had ceased. The reaction was usually terminated at about 2.5 hours. The rate of pressure drop and the composition of the product were then determined and recorded. A comparison of the pressure drop is shown in the graph identified as Propylene Oxidation: O₂ Consumption Rate Comparison. In the graph, are shown the reactor pressure vs. time in hours curves for propylene oxidation in water, 50% aqueous propionic acid, 75% aqueous butyric acid, 80% aqueous valeric acid, 75% aqueous acetone, and 75% aqueous methyl isobutyl ketone. The concentrations are varied to maintain a single phase reaction solvent.

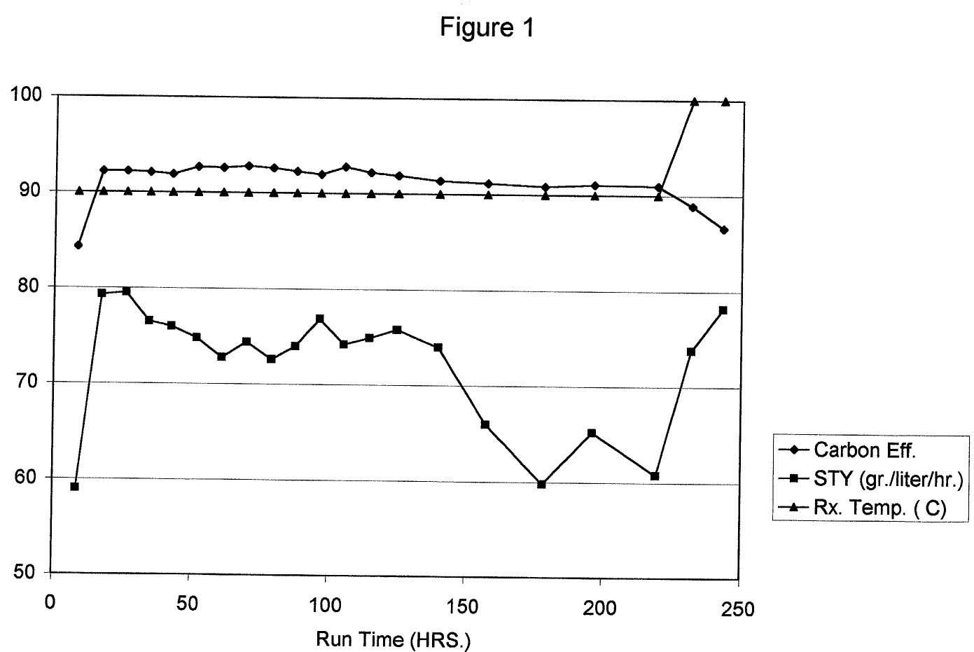

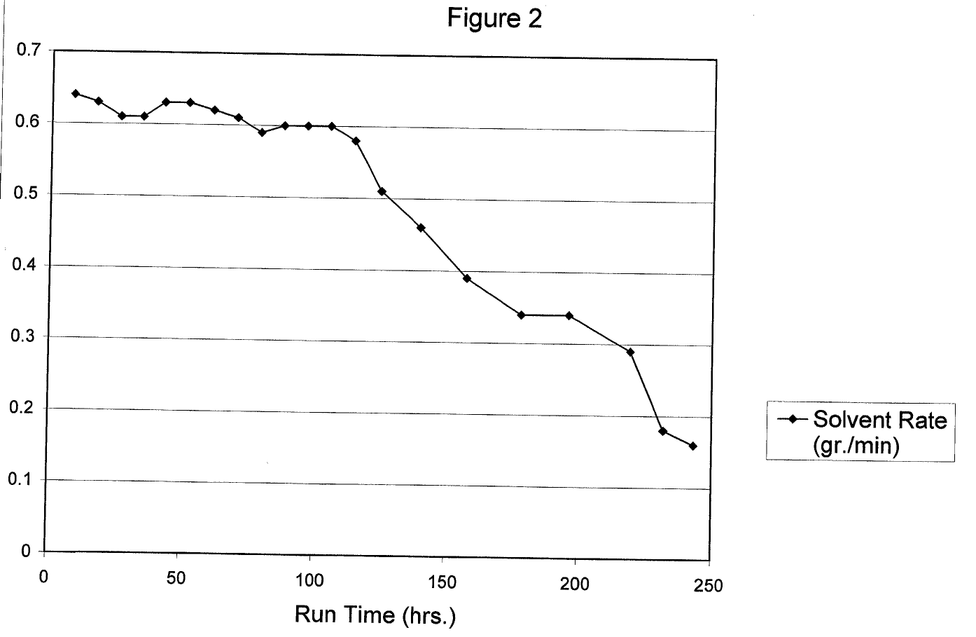

The following example of the manufacture of acrylic acid, which is not to be considered limiting to the scope of the invention, describes a continuous run of over 200 hours. In a continuous reactor with a total volume of 300 ml were placed 10 g of palladium acetate, 132 ml of valeric acid, and 18 ml of water. The reactor was swept with propylene several times to remove any air and then pressured with propylene to an internal pressure of 7.8 bar. The reactor was heated to 80° C. and the contents were stirred for one hour. At that time a continuous stream of propylene and air (equimolar amounts of propylene and oxygen) was admitted and the pressure was raised to 455 psig (32 bar). The temperature was held at 90° C. until near the end of the run where the temperature was increased to 100° C. Filter plugging was noted about half way through the run, which required a reduced solvent flow and reduced the STY for the system. The propylene partial pressure in the reactor was about 45 psig (3.1 bar) based on the concentration of propylene in the vent gas. The reaction operated at about a 39% conversion of the oxygen. In the figures, FIG. 1 shows the carbon efficiency and STY of the reaction. Carbon efficiency is shown in percent based upon weight of carbon incorporated in the acrylic acid formed and the propylene converted to acrylic acid as determined by chromatographic analysis of the product. FIG. 2 shows the solvent recycle rate in grams per minute for the continuous run.

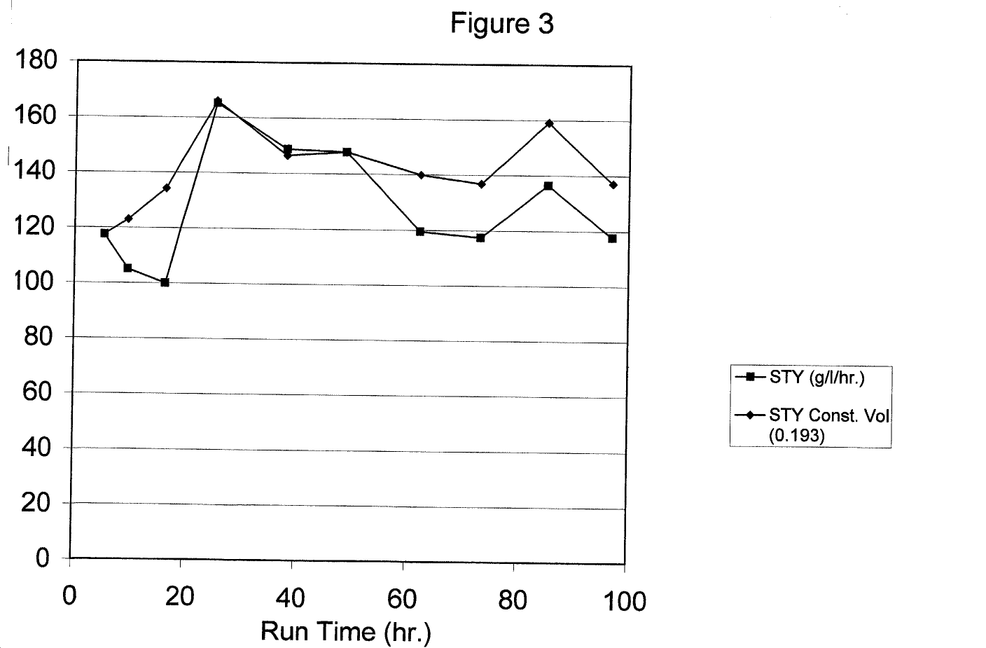

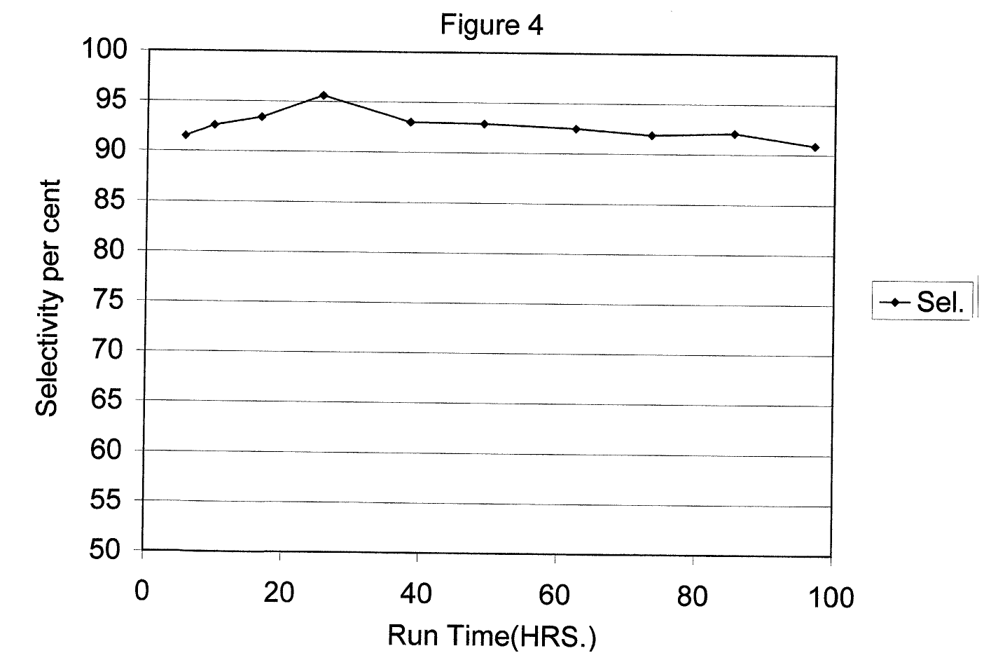

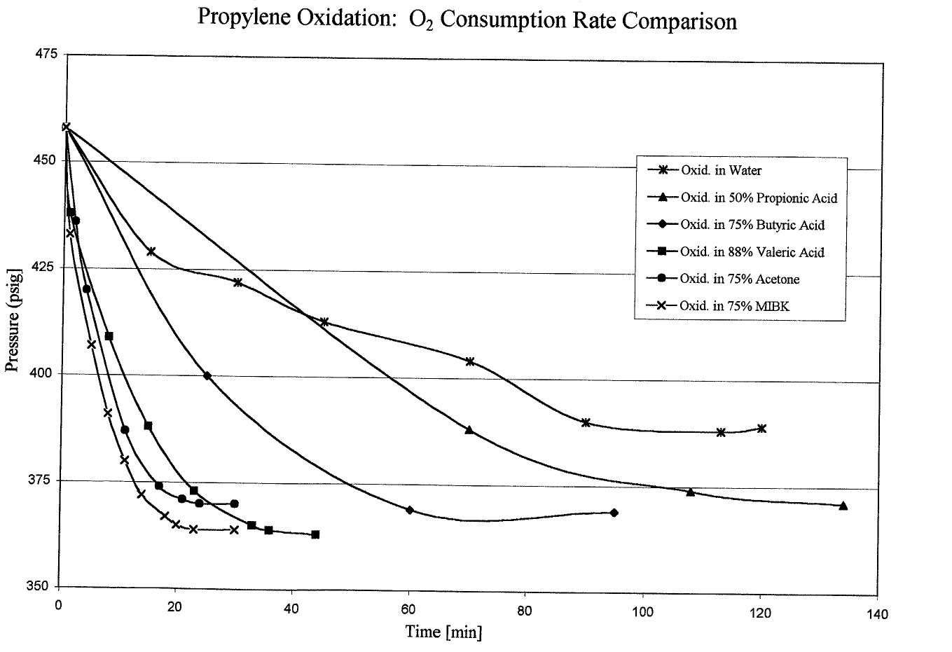

The following example of the manufacture of methacrylic acid, which is not to be considered limiting to the scope of the invention, describes a continuous run of 100 hours. In the reactor as described above methyl isobutyl ketone with 20% water loading was used as the solvent mixture, the pre-reduced (with propylene) palladium loading was 4.4%, temperature was 90° C., and pressure was 455 psig. Air was introduced at 2.5 std liters/minute and isobutylene as liquid at 0.86 g/min. The mixture was stirred at 2000 rpm. FIG. 3 identifies the STY in grams/liter/hr and a Constant Volume STY. FIG. 4 is a plot of the selectivity to a mixture containing methacrolein, methacrylic acid, and esters as determined by vapor phase chromatography during the continuity of the run.

What is claimed is:

1. A method for the manufacture of acrylic acid or methacrylic acid which comprises: a) continuously reacting oxygen with propylene or isobutylene in the presence of an unsupported palladium catalyst suspended in an aqueous solvent containing as a co-solvent a maximum concentration of a C₂-C₆ carboxylic acid or C₃-C₆ ketone, b) recovering the acrylic acid or methacrylic acid formed, and d) recycling the solvent to the reactor.

2. The method of claim 1 wherein the propylene to oxygen ratio is above about 1:1.

3. The method of claim 2 wherein the propylene to oxygen ratio is from about 1:1 to about 1:5.

4. The method of claim 1 wherein the reaction is carried out at from 50°-150° C.

5. The method of claim 4 wherein the reaction is carried out at from 60°-90° C.

6. The method of claim 1 wherein the reaction is carried out at from 1-50 bar.

7. The method of claim 1 wherein the co-solvent is propionic acid.

8. The method of claim 1 wherein the co-solvent is valeric acid.

9. The method of claim 1 wherein the co-solvent is butyric acid.

10. The method of claim 1 wherein the co-solvent is acetone.

11. The method of claim 1 wherein the co-solvent is methyl isobutyl ketone.

12. A method for the manufacture of acrylic acid by the oxidation of propylene by: a) reducing palladium acetate to unsupported palladium with propylene in an oxygen-free single or two-phase aqueous solvent containing as a co-solvent a maximum concentration of a C₂-C₆ carboxylic acid or C₃-C₆ ketone, b) thereafter adding oxygen and propylene in a continuous manner, c) recovering the acrylic acid formed, and d) recycling the aqueous solvent to the reactor.

13. The method of claim 12 wherein the propylene to oxygen ratio is above about 1:1.

14. The method of claim 13 wherein the propylene to oxygen ratio is from about 1:1 to about 1:5.

15. The method of claim 12 wherein the reaction is carried out at from 50°-150° C.

16. The method of claim 15 wherein the reaction is carried out at from 60°-90° C.

17. The method of claim 12 wherein the reaction is carried out at from 1-50 bar.

18. The method of claim 12 wherein the co-solvent is propionic acid.

19. The method of claim 12 wherein the co-solvent is valeric acid.

20. The method of claim 12 wherein the co-solvent is butyric acid.

21. The method of claim 12 wherein the co-solvent is acetone.

22. The method of claim 12 wherein the co-solvent is methyl isobutyl ketone.

23. The method of claim 1 for the manufacture of acrylic acid which comprises:
a) continuously reacting oxygen with propylene in the presence of an unsupported palladium catalyst suspended in an aqueous solvent containing as a co-solvent a maximum concentration of a C₂-C₆ carboxylic acid or C₃-C₆ ketone,
b) recovering the acrylic acid formed, and
c) recycling the solvent to the reactor.

24. The method of claim 23 wherein the propylene to oxygen ratio is above about 1:1.

25. The method of claim 24 wherein the propylene to oxygen ratio is from about 1:1 to about 1:5.

26. The method of claim 23 wherein the reaction is carried out at from 50°-150° C.

27. The method of claim 26 wherein the reaction is carried out at from 60°-90° C.

28. The method of claim 23 wherein the reaction is carried out at from 1-50 bar.

29. The method of claim 23 wherein the co-solvent is propionic acid.

30. The method of claim 23 wherein the co-solvent is valeric acid.

31. The method of claim 23 wherein the co-solvent is butyric acid

32. A method for the manufacture of methacrylic acid by the oxidation of isobutylene by:
a) reducing palladium acetate to unsupported palladium with propylene in an oxygen-free single or two-phase aqueous solvent containing as a co-solvent a maximum concentration of a C₂-C₆ carboxylic acid,
b) thereafter adding air and propylene in a continuous manner,
c) recovering the acrylic acid formed, and
d) recycling the aqueous solvent to the reactor.

33. The method of claim 33 wherein the isobutylene to oxygen ratio is above about 1:1.

34. The method of claim 33 wherein the isobutylene to oxygen ratio is from about 1:1 to about 1:5.

35. The method of claim 33 wherein the reaction is carried out at from 50°-150° C.

36. The method of claim 35 wherein the reaction is carried out at from 60°-90° C.

37. The method of claim 33 wherein the reaction is carried out at from 1-50 bar.

38. The method of claim 33 wherein the co-solvent is methyl isobutyl ketone.

39. The method of claim 33 wherein the co-solvent is acetone.

40. The method of claim 33 wherein the co-solvent is methyl ethyl ketone.

41. The method of claim 1 for the manufacture of methacrylic acid which comprises: a) continuously reacting oxygen with isobutylene in the presence of an unsupported palladium catalyst suspended in an aqueous solvent containing as a co-solvent a maximum concentration of a C₂-C₆ carboxylic acid or C₃-C₆ ketone, b) recovering the methacrylic acid formed, and d) recycling the solvent to the reactor.

42. The method of claim 41 wherein the isobutylene to oxygen ratio is above about 1:1.

43. The method of claim 42 wherein the isobutylene to oxygen ratio is from about 1:1 to about 1:5.

44. The method of claim 41 wherein the reaction is carried out at from 50-150° C.

45. The method of claim 41 wherein the reaction is carried out at from 1-50 bar.

46. The method of claim 41 wherein the co-solvent is acetone.

47. The method of claim 41 wherein the co-solvent is methyl isobutyl ketone.

48. A method for the manufacture of acrylic acid by the oxidation of acrolein by: a) reducing palladium acetate to unsupported palladium with propylene in an oxygen-free single or two-phase aqueous solvent containing as a co-solvent a maximum concentration of a C₂-C₆ carboxylic acid, b) thereafter adding air and propylene in a continuous manner, c) recovering the acrylic acid formed, and d) recycling the aqueous solvent to the reactor.

49. A palladium catalyst useful for the oxidation of propylene to acrylic acid is manufactured by the reduction of palladium acetate with propylene in a single or two-phase aqueous solution containing as a co-solvent a maximum concentration of a C₂-C₆ carboxylic acid or C₃-C₆ ketone.

50. A palladium catalyst useful for the oxidation of isobutylene to methacrylic acid is manufactured by the reduction of palladium acetate with propylene in a single or two-phase aqueous solution containing as a co-solvent a maximum concentration of a C₂-C₆ carboxylic acid or C₃-C₆ ketone.
